(19) United States
(12) Patent Application Publication
(10) Pub. No.: US 2006/0127327 A1
(43) Pub. Date: Jun. 15, 2006

(54) Monoclonal antibodies specific for cariogenic bacteria

(76) Inventors: Wenyuan Shi, Los Angeles, CA (US); Fang Gu, Los Angeles, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/531,411

(22) PCT Filed: Oct. 14, 2003
(86) PCT No.: PCT/US03/32543

§ 371 Date: Nov. 4, 2005

Related U.S. Application Data
(60) Provisional application No. 60/418,768, filed on Oct. 15, 2002.

(51) Int. Cl. A61K 39/40; A61K 8/96; G01N 33/554; C07K 16/12

(52) U.S. Cl. 424/50; 435/7.32; 530/388.4; 424/164.1

(57) ABSTRACT

Antibodies, as well as binding fragments and mimetics thereof, that specifically bind to Actinomyces or Lactobacillus cariogenic bacteria are provided. The subject binding agents, e.g., antibodies, fragments and mimetics thereof, etc., are characterized in that they are highly sensitive and specific for their target bacteria. Also provided are methods and devices for screening samples for the presence of cariogenic bacteria. In addition therapeutic treatment protocols and compositions are provided.

### FIELD OF THE INVENTION

The field of this invention is dental caries.

### BACKGROUND OF THE INVENTION

Dental caries is a chronic infectious disease resulting from a complex interaction of microflora, environmental factors (such as diet) and host. Recent epidemiologic studies indicate that caries risk is not evenly distributed in the general population. It has been estimated that one-quarter of school-aged children experience three-quarter of dental decay. This skewed distribution of disease calls for methods to identify those at greatest risk. Although a number of factors, such as diet, salivary flow, and fluoride level contribute to dental caries, the disease is absolutely dependent on the presence of a few groups of cariogenic bacteria (e.g., mutans streptococci, lactobacilli and actinomyces). These bacteria colonize the surfaces or roots of teeth and produce acids that dissolve tooth mineral. As the disease progresses, they invade the softened teeth and the destructive process continues. Epidemiological studies indicate a possible association between the level and proportion of cariogenic bacteria in saliva or plaque and the incidence of dental caries. This association suggests that with proper bacterial detection methods, people with high risk for dental caries might be diagnosed by determining the level and proportions of caries-related bacteria in plaque or, more conveniently, in saliva.

Literature

Of interest are U.S. Pat. No. 6,231,857 and international publication nos. WO 00/11037 and WO 02/15931. Thunheer et al. (1997) FEMS Microbiol. Lett. (1987) Scand. J. Dent Res. 95:136-143; Ellen (1976) Infect Immun. 14:1119-1124;

### SUMMARY OF THE INVENTION

Antibodies, as well as binding fragments and mimetics thereof, that specifically bind to Actinomyces or Lactobacillus cariogenic bacteria are provided. The subject binding agents, e.g., antibodies, fragments and mimetics thereof, etc., are characterized in that they are highly sensitive and specific for their target bacteria. Also provided are methods and devices for screening samples for the presence of cariogenic bacteria. In addition, therapeutic treatment protocols and compositions are provided.

### BRIEF DESCRIPTION OF THE FIGURES

FIGS. 1A-H depict flow cytometry analysis of oral bacteria.

FIGS. 2A and 2B depict distribution of salivary A. naeslundii (genospecies 1) and L. casei counts within a human population. The data were based on saliva samples collected from 100 children aged from 2-16. The unstimulated saliva samples were collected and fixed at the dentists' chairside and shipped to UCLA for processing (as described in MATERIALS AND METHODS). (a) Distribution of salivary A. naeslundii (genospecies 1) counts; (b) Distribution of salivary L. casei counts.

### DESCRIPTION OF THE SPECIFIC EMBODIMENTS

Antibodies, as well as binding fragments and mimetics thereof, that specifically bind to Actinomyces or Lactobacillus cariogenic bacteria are provided. The subject binding agents, e.g., antibodies, fragments and mimetics thereof, etc., are characterized in that they are highly sensitive and specific for their target bacteria. Also provided are methods and devices for screening samples for the presence of cariogenic bacteria. In addition, therapeutic treatment protocols and compositions are provided.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The invention describes two species-specific monoclonal IgG antibodies, referred to as SWLA4 and SWLA5, which recognize a species-specific epitope on the cell surface of A. naeslundii and L. casei respectively. More specifically, SWLA4 specifically recognizes A. naeslundii genospecies 1 (and not A. naeslundii genospecies 2), and thus allows detection of A. naeslundii genospecies 1. The present invention provides a subject antibody immobilized on an insoluble support, e.g., a plate, a bead, etc. The present invention further provides a panel of immobilized antibodies, wherein each antibody is specific for a cariogenic bacterium, and wherein the panel includes SWLA4 and/or SWLA5; and at least one additional antibody specific for a cariogenic bacterium. The invention includes methods of using the monoclonal antibodies and analogous agents to detect quantity and presence of target bacteria to monitor the onset and severity of dental caries.

Antibodies

A subject antibody binds specifically to a cariogenic bacterium. In many embodiments, a subject antibody is substantially isolated. A “substantially isolated” or “isolated” antibody is one that is substantially free of the macromolecules with which it is associated in nature. By substantially free is meant at least 50%, preferably at least 70%, more preferably at least 80%, and even more preferably at least 90% free of the materials with which it is associated in nature.

The term “binds specifically,” in the context of antibody binding, refers to high avidity and/or high affinity binding of an antibody to a specific cariogenic bacterium, e.g., to an epitope on a cariogenic bacterium. Antibody binding to an epitope on a specific cariogenic bacterium is preferably stronger than binding of the same antibody to any other epitope, particularly those which may be present in molecules in association with, or in the same sample, as the specific cariogenic bacterium of interest, e.g., binds more strongly to an epitope on a specific cariogenic bacterium than to an epitope on a different cariogenic bacterium, or, e.g., binds more strongly to an epitope on a specific genospecies of a cariogenic bacterium than to an epitope on a different genospecies of the cariogenic bacterium, so that by adjusting binding conditions the antibody binds almost exclusively to the specific epitope on the specific cariogenic bacterium and not to any other cariogenic bacteria. Antibodies which bind specifically to a given cariogenic bacterium may be capable of binding other cariogenic bacteria at a weak, yet detectable, level (e.g., 10% or less of the binding shown to the cariogenic bacterium of interest). Such weak binding, or background binding, is readily discernible from the specific antibody binding to a specific cariogenic bacterium, e.g. by use of appropriate controls. In general, antibodies of the invention which bind to a specific cariogenic bacterium with a binding affinity of 10⁻⁷ M or more, preferably 10⁻⁸ M or more (e.g., 10⁻⁹ M, 10⁻¹⁰, 10⁻¹¹, etc.). In general, an antibody with a binding affinity of 10⁻⁶ M or less is not useful in that it will not bind an antigen at a detectable level using conventional methodology currently used.

In some embodiments, a subject antibody comprises a detectable label. Suitable detectable labels include, but are not limited to, radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I); fluorescent labels, fluorescein isothiocyanate (FITC), rhodamine, lanthanide phosphors, Texas Red, phycoerythrin, allophycocyanin, and fluorescent proteins; magnetic particles; enzymatic labels (e.g., horseradish peroxidase, β-galactosidase, luciferase, alkaline phosphatase); chemiluminescent labels; biotinyl groups; predetermined polypeptide epitopes recognized by a secondary reporter (e.g., leucine zipper pair sequences; binding sites for secondary antibodies; metal binding domains; epitope tags, including, but not limited to, hemagglutinin, FLAG and the like); specific binding molecules; and the like. Specific binding molecules include pairs, such as biotin and streptavidin, digoxin and antidigoxin etc. Suitable fluorescent proteins include those described in Matz et al. ((1999) Nature Biotechnology 17:969-973), a green fluorescent protein from any species or a derivative thereof; e.g., a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); a GFP from Aequoria victoria or fluorescent mutant thereof, e.g., as described in U.S. Pat. Nos. 6,066,476; 6,020,192; 5,985,577; 5,976,796; 5,968,750; 5,968,738; 5,958,713; 5,919,445; 5,874,304. In some embodiments, labels are attached by spacer arms of various lengths to reduce potential steric hindrance.

In some embodiments, a subject antibody is immobilized on an insoluble support. Suitable insoluble supports include plastic plates (e.g., 96-well plates, microtiter plates, and the like); beads, e.g., polystyrene beads, magnetic beads, and the like; membranes, e.g., polyvinylpyrrolidone, nitrocellulose membranes, and the like; test strips; dip sticks; silicon chips; and the like. Antibodies immobilized on substrates for diagnostic purposes are described in the art. See, e.g., Holt et al. (2000) Nucl. Acids Res. 28:E72; and de Wildt et al. (2000) Nat Biotechnol. 18:989-994. Thus, the present invention provides an insoluble support having immobilized thereon a subject antibody. In some embodiments, a subject insoluble support comprises two or more antibodies, each having specificity for a different cariogenic bacterium. In some embodiments, a subject insoluble support comprises SWLA4 immobilized on the support. In other embodiments, a subject insoluble support comprises SWLA5 immobilized on the support. In other embodiments, a subject insoluble support comprises SWLA4 and SWLA5. In any of these embodiments, a subject insoluble support will further comprise at least one additional antibody specific for a cariogenic bacterium other than L. casei or A. naeslundi genospecies 1. For example, a subject insoluble support may further comprise an antibody specific for S. mutans.

Because the monoclonal antibodies of the invention are able to detect low numbers of target bacteria in small samples they are able to be used to screen for target bacteria. These monoclonal antibodies also permit the development of simple and inexpensive dental caries detection methods that could be used for caries risk assessment at a dentist's chairside or in the patient's household.

The most preferred antibodies will selectively bind to target bacteria and will not bind (or will bind weakly) to non-target bacteria. The antibodies that are particularly contemplated include monoclonal antibodies as well as fragments of monoclonal antibodies containing a target bacteria antigen-binding domain. The invention also encompasses antibody fragments that specifically recognize the target bacteria. As used herein, an antibody fragment is defined as at least a portion of the immunoglobulin molecule which binds to its target, i.e., the antigen binding region on the target bacteria. This includes Fv, Fab, Fab′ and F(ab)′₂ fragments of appropriate specificity.

The invention further includes a monoclonal antibody that specifically binds an antigen found on the surface of a target bacterium. The antigen that is bound is one of those bound by at least one of the monoclonal antibody produced by a hybridoma designated SWLA4, or the monoclonal antibody produced by a hybridoma designated SWLA5.

Methods for the preparation of human monoclonal antibodies are known in the art and include phage display techniques and isolation of human hybridomas using B lymphocytes from patients producing antibodies against target bacterium, as well as in vitro immunization techniques. Such techniques are well known in the art and are described, for example, in C. A. K. Borrebaeck, ed., “Antibody Engineering” (2d ed. Oxford University Press, New York, 1995), incorporated herein by this reference.

The invention further includes chimeric antibodies, including humanized antibodies. This includes chimeric antibodies that have complementarity-determining regions that are identical with the complementarity-determining regions of one of:

(a) a monoclonal antibody produced by a hybridoma designated SWLA4; or

(b) a monoclonal antibody produced by a hybridoma designated SWLA5.

Also within the scope of the invention are chimeric antibodies that have complementarity-determining regions that are identical with the complementarity-determining regions of an antibody that binds an antigen on the surface of target bacterium and can compete at least about 80% as effectively on a molar basis with at least one of SWLA4 or SWLA5 for binding to the antigen on the surface of target bacterium.

These chimeric antibodies specifically bind an antigen on the surface of target bacterium and which have at least a portion of the amino acid sequence of the heavy chain or the light chain of a different species origin than the species origin of the complementarity-determining regions. In one alternative, at least a portion of the amino acid sequence of the heavy chain or the light chain is of human origin so that the chimeric antibody is a humanized antibody. In a version of this alternative, substantially all of the amino acid sequences of the heavy chain and the light chain outside the complementarity-determining regions are of human origin.

As indicated, chimeric antibodies according to the present invention may have a non-human antigen-binding site and a humanized effector binding region. The non-human antigen-binding portion may include, but is not limited to, a murine, canine, feline or other veterinary model or other mammalian antigen-binding site.

Methods for producing chimeric antibodies, including humanized antibodies, are well known in the art and are described, for example, in C. A. K. Borrebaeck, ed., “Antibody Engineering” (2d ed., Oxford University Press, New York, 1995), incorporated herein by this reference.

The invention further includes single-chain binding fragments, known generally as sFv, that have the appropriate specificity for the antigen on the cell surface of target bacteria as defined above. Methods for preparing such sFv are generally known in the art and are described, for example, in C. A. K. Borrebaeck, ed., “Antibody Engineering” (2d ed., Oxford University Press. New York, 1995), incorporated herein by this reference.

Preparation of Antibodies

The monoclonal antibodies SWLA4 and SWLA5 can be prepared by hybridoma fusion techniques or by techniques that utilize EBV-immortalization technologies. Hybridoma fusion techniques were first introduced by Kohler and Milstein (see, Kohler and Milstein, (1975); Brown et al., (1981); Brown et al., (1980); Yeh et al., (1976); and Yeh et al., (1982)).

These techniques involve the injection of an immunogen (e.g., purified antigen or cells or cellular extracts carrying the antigen) into an animal (e.g., a mouse) so as to elicit a desired immune response (i.e., production of antibodies) in that animal. For example, the target bacterium in whole cell form may be used as the immunogen. In the illustrative example herein, whole cell target bacteria were used as the immunogen. The cells are injected repeatedly, for example, into a mouse and, after a sufficient time, the mouse is sacrificed and somatic antibody-producing cells are obtained. The use of other mammalian models, for example rat, rabbit, etc.; and non-mammalian models, e.g., frog somatic cells, is also possible. The cell chromosomes encoding desired immunoglobulins are immortalized by fusing them with myeloma cells, generally in the presence of a fusing agent such as polyethylene glycol (PEG). Any of a number of myeloma cell lines may be used as a fusion partner according to standard techniques; for example, the P3-NSI/1-Ag4-1, P3-x63-Ag8.653 or Sp2/0-Ag14 myeloma lines. These myeloma lines are available from the American Type Culture Collection (ATCC), in Rockville, Md.

The resulting cells, which include the desired hybridomas, are then grown in a selective medium, such as HAT medium, in which unfused parental myeloma or lymphocyte cells eventually die. Only the hybridoma cells survive and can be grown under limiting dilution conditions to obtain isolated clones. The supernatants of the hybridomas are screened for the presence of antibody of the desired specificity, e.g., by immunoassay techniques using the antigen that has been used for immunization. Positive clones can then be subcloned under limiting dilution conditions and the monoclonal antibody produced can be isolated. Various conventional methods exist for isolation and purification of the monoclonal antibodies so as to free them from other proteins and other contaminants. Commonly used methods for purifying monoclonal antibodies include ammonium sulfate precipitation, ion exchange chromatography, and affinity chromatograhy (Zola et al. (1982)). Hybridomas produced according to these methods can be propagated in vitro or in vivo (in ascites fluid) using techniques known in the art (see, generally, Fink et al., supra, 1984).

Generally, the individual cell line may be propagated in vitro, for example in laboratory culture vessels, and the culture medium containing high concentrations of a single specific monoclonal antibody can be harvested by decantation, filtration or centrifugation. Alternatively, the yield of monoclonal antibody can be enhanced by injecting a sample of the hybridoma into a histocompatible animal of the type used to provide the somatic and myeloma cells for the original fusion. Tumors secreting the specific monoclonal antibody produced by the fused cell hybrid develop in the injected animal. The body fluids of the animal, such as ascites fluid or serum, provide monoclonal antibodies in high concentrations. When human hybridomas or EBV-hybridomas are used, it is necessary to avoid rejection of the xenograft injected into animals such as mice. Immunodeficient or nude mice may be used or the hybridoma may be passaged first into irradiated nude mice as a solid subcutaneous tumor, cultured in vitro and then injected intraperitoneally into pristane primed, irradiated nude mice which develop ascites tumors secreting large amounts of specific human monoclonal antibodies.

For certain therapeutic applications chimeric (mouse-human) or human monoclonal antibodies may be preferable to murine antibodies, because patients treated with mouse antibodies generate human antimouse antibodies. (Shawler et al., (1985)). Chimeric mouse-human monoclonal antibodies reactive with the target bacteria can be produced, for example, by techniques developed for the production of chimeric antibodies (Oi et al., (1986); Liu et al., (1987)). Accordingly, genes coding for the constant regions of the SWLA4 or SWLA5 antibody molecule are substituted with human genes coding for the constant regions of an antibody with appropriate biological activity (such as the ability to selectively bind the target bacterium of the present invention).

Novel antibodies of mouse or human origin can be also made that are analogous to the SWLA4 or SWLA5 antibody and that have the appropriate biological functions. These antibodies can have complementarity-determining regions (CDRs) that are identical to one of SWLA4 or SWLA5. Alternatively, these antibodies can bind an antigen on the surface of a target bacterium of the present invention and can compete at least about 80% as effectively on a molar basis with at least one of SWLA4 or SWLA5 for binding to the antigen on the surface of target bacterium. These antibodies have substantially no reactivity with any of the non target bacterial strains listed in Table 1, below. Preferably, the monoclonal antibody competes at least about 90% as effectively on a molar basis.

For example, human monoclonal antibodies may be made by using the antigen, e.g. the portion of the cell surface of the target bacterium which binds the antibodies SWLA4 or SWLA5 of the invention, to sensitize human cells to the antigen in vitro followed by EBV-transformation or hybridization of the antigen-sensitized cells with mouse or human cells, as described by Borrebaeck et al. (1988).

Methods

The specificity of the SWLA4 and SWLA5 antibodies for their target bacteria antigen make these antibodies excellent markers for screening, diagnosis, prognosis, and follow-up assays, imaging methodologies, and therapeutic methods in the management of dental caries.

In many embodiments, a subject method involves contacting a biological sample with a subject antibody; and detecting specific binding between the subject antibody and molecules in the biological sample. A “biological sample” encompasses a variety of sample types obtained from an individual (e.g., biological fluids, biological tissues) and can be used in a diagnostic or monitoring assay. In many embodiments, a biological sample is saliva, or other oral or dental tissue or secretions. The definition also includes samples that have been manipulated in any way after their procurement, such as by treatment with reagents, solubilization, or enrichment for certain components, such as certain bacteria.

The invention provides various immunological assays useful for the detection of target bacteria and for the diagnosis of dental caries or the risk thereof. This includes various immunological assay formats well known in the art, including, but not limited to, various types of radioimmunoassays, enzyme-linked immunosorbent assays (ELISA), enzymelinked immunofluorescent assays (ELIFA), and the like. In addition, immunological imaging methods capable of detecting dental caries are also provided by the invention, including but not limited to a colloidal-gold based calorimetric assay, and radioscintigraphic imaging methods using radiolabeled SWLA4 and SWLA5 antibodies (e.g., U.S. Pat. No. 4,920,059 issued Apr. 24, 1990; U.S. Pat. No. 5,079,172 issued Jan. 7, 1992). In addition the antibodies of the invention can be conjugated with other dyes or fluorescent markers and used directly on the tooth to image caries. Such assays may be clinically useful in the detection and monitoring of dental caries. Such assays generally comprise using one or more of the SWLA 4 and SWLA 5 antibodies of the present invention, and in some embodiments in conjunction with the SWLA1, SWLA2, and SWLA3 antibodies disclosed in U.S. Pat. No. 6,231,857, the disclosure of which is herein incorporated by reference.

In addition to the immunological assays and imaging methods, the invention also includes an immunoconjugate comprising a molecule containing the antigen-binding region of the SWLA4 or SWLA5 antibody, or a fragment thereof containing the antigen binding region, joined to for example a therapeutic agent, a diagnostic agent or a cytotoxic agent for treatment of dental caries. Examples of cytotoxic agents include, but are not limited to, chlorhexidine, fluoride, ricin, doxorubicin, daunorubicin, taxol, ethidium bromide, mitomycin, etoposide, tenoposide, vincristine, vinblastine, colchicine, dihydroxyanthracenedione, actinomycin D, diphtheria toxin, Pseudomonas exotoxin (PE) A, PE40, abrin, glucocorticoid and radioisotopes.

The SWLA4, and SWLA5 monoclonal antibodies of the invention are useful for diagnostic applications, both in vitro and in vivo, for the detection of dental caries. In vitro diagnostic methods are well known in the art (see, e.g., Roth, supra 1986, and Kupchik, supra 1988), and include immunohistological detection of dental caries or serologic detection of target bacteria (e.g., in saliva samples or other biological fluids).

Immunohistological techniques involve contacting a biological specimen, such as a saliva, tartar, or plaque specimen, with the antibody of the invention and then detecting the presence in the specimen of the antibody complexed to its antigen. The formation of such antibody-antigen complexes with the specimen indicates the presence of the antigen, target bacterium. Detection of the antibody in the specimen can be accomplished using techniques known in the art, such as the immunoperoxidase staining technique, the avidin-biotin (ABC) technique or immunofluorescence techniques (Ciocca et al., (1986); Helistrom et al., (1986); and Kimball (ed.,), (1986)).

Serologic diagnostic techniques involve the detection and quantitation of target bacterium antigens that have been secreted or “shed” into the saliva or other biological fluids of patients with dental caries. Such antigens can be detected in the saliva using techniques known in the art such as radioimmunoassays (RIA) or enzyme-linked immunosorbent assays (ELISA) wherein an antibody reactive with the “shed” antigen is used to detect the presence of the antigen in a fluid sample (see, e.g., Uotila et al., (1981) and Allum et al., 1986). These assays, using the antibodies disclosed herein, can therefore be used for the detection of target bacteria in biological fluids. Thus, it is apparent from the foregoing that the antibodies of the invention can be used in most assays involving antigen-antibody reactions. These assays include, but are not limited to, standard RIA techniques, both liquid and solid phase, as well as ELISA assays, immunofluorescence techniques, and other immunocytochemical assays (see, e.g., Sikora et al. (1984)).

The antibodies of the invention are also useful for in vivo diagnostic applications for the detection of dental caries. One such approach involves the detection of dental caries in vivo by imaging techniques using the antibody labeled with an appropriate imaging reagent that produces a detectable signal when bound to target bacterium. Imaging reagents and procedures for labeling antibodies with such reagents are well known (see, e.g., Wensel and Meares, (1983); Colcher et al., (1986)). The labeled antibody may be detected by a technique such as radionuclear scanning (see, e.g., Bradwell et al. (1985)).

The antibody fragments used in the immunoconjugates can include Fv, Fab, Fab′ or F(ab)′₂ fragments. Use of immunologically reactive fragments, such as the Fv, Fab, Fab′, or F(ab)′₂ fragments, is often preferable, especially in a therapeutic context, as these fragments are generally less immununogenic than the whole immunoglobulin. These antibodies, as well as unconjugated antibodies, may be useful therapeutic agents naturally targeted to target bacerial cells to kill the cells, thus preventing and or treating dental caries resulting from the accumulation of target bacteria. Techniques for conjugating therapeutic agents to antibodies are well known (see, e.g., Arnon et al., 1985; Hellstrom et al. 1987; Thorpe, (1985); and Thorpe et al., (1982)).

The SWLA4 and SWLA5 antibodies may also be used in methods for purifying target bacterial proteins and peptides and for isolating homologues and related molecules. Methods for purification of proteins and peptides using antibodies as capture reagents are well known in the art. For example, in one embodiment, a method of purifying a target bacterial protein comprises incubating a SWLA4 or SWLA5 antibody, which has been coupled to a solid matrix, with a lysate or other solution containing target bacteria proteins or peptides, under conditions which permit the SWLA4 or SWLA5 antibody to bind to the target bacterial protein or peptides; washing the solid matrix to eliminate impurities; and eluting the target bacterial proteins or fragments from the coupled antibody.

The invention further includes a method for detecting the presence of target bacteria on teeth in a subject or in a saliva, plaque, or tartar sample from a subject, comprising contacting at least one tooth or the sample with the SWLA4 or SWLA5 antibody and detecting the binding of the antibody with the target bacteria on the tooth and or in the sample. The antibody can be administered by topical application to the surface of the teeth by means including in a toothpaste, mouthwash, lozenge, gel, powder, spray, liquid, tablet, or chewing gum. One can detect the presence of target bacteria by determining the presence of a complex formed between the monoclonal antibodies and target bacteria cells as a result of contacting the tooth and or the sample with a labeled antibody, the complex being indicative of the presence of target bacteria in the sample. The antibodies of the invention can be labeled so as to directly or indirectly produce a detectable signal. The label can for example be selected from the following compounds a radiolabel, an enzyme, a chromophore, a chemiluminescent moiety, a bioluminescent moiety, or a fluorescer. When a fluorescer is used the fluorescence can be detected by means of fluorescence microscopy, fluorometer, or by flow cytometry. A colloidal gold calorimetric system can also be used to detect the presence of target bacteria. The colloidal gold system is Well known in the art. (J. A. K. Hasan, et al. (1994); and E. Harlow, D. Lane. (1988)).

The invention also includes a method for diagnosing, in a subject, the early onset of dental caries. This can be accomplished by quantitatively determining on at least one tooth in a subject, or in a saliva, plaque, or tartar sample from a subject, the number of target bacteria present using an antibody of the invention and comparing the number of target bacterial cells so determined to the amount in a sample from a normal control, i.e. a subject free from dental caries. The normal range for target bacteria can be determined using any of the above detection methods (i.e. detecting labeled antibody to target bacteria) and quantifying the amount of target bacteria in a normal subject or subjects free of dental caries. For example, a normal range can be 1 cell/ml to approximately 1×10⁵ cells/ml or 1×10⁵ cells/ml to 1×10⁶ cell/ml. Other ranges are possible. If the subject has a measurably higher amount of target bacteria present that is outside of the normal range it would indicate the early onset of dental caries in the subject.

The invention also includes a method for monitoring the course of dental caries in a subject. One can test teeth or a saliva, plaque, or tartar sample from a subject with the antibodies of the invention at different points in time and determine if there has been a change in the level of target bacteria present. An increase over a previous reading for that individual would suggest increased caries activity. For example if a first test of a subject's saliva sample gave a result of less than 1×10⁵ target bacterial cells/ml and a sample taken at a later time gave a result of greater than 1×10⁻⁵ target bacterial cells/ml it would indicate that the subject now has an increased risk of dental caries.

The invention further comprises a method of protecting teeth from dental caries by topically applying an SWLA4 or SWLA5 antibody, or a fragment thereof containing the target bacterial antigen binding activity, to teeth of a subject. The antibody can be applied topically to the surface of the teeth by means of for example, of a toothpaste, mouthwash, lozenge, gel, powder, spray, liquid, tablet, or chewing gum formulated using standard methods. The antibody can be linked to a toxic agent that kills the bacteria and applied to the surface of the teeth by, for example, any of the above methods. The proper dose of the monoclonal antibodies of the invention can be easily determined using methods which are well known to one skilled in the art (see, generally, Goodman et al. (ed.), 1993).

Kits

The methods described herein for detecting target bacteria may be performed using diagnostic kits (e.g., U.S. Pat. No. 5,141,850 issued Aug. 25, 1992; U.S. Pat. No. 5,202,267 issued Apr. 13, 1993; U.S. Pat. No. 5,571,726 issued Nov. 5, 1996; U.S. Pat. No. 5,602,040 issued Feb. 11, 1997). Such kits include at least one monoclonal antibody of the invention and reagents for detecting the binding of the monoclonal antibody to target bacterial cells present on teeth in or in a sample, e.g. of saliva, taken from a subject. The reagents include agents capable of detection, for example by fluorescence and ancillary agents such as buffering agents. The kits may also include an apparatus or container for conducting the methods of the invention and/or for transferring samples to a diagnostic laboratory for processing, as well as suitable instructions for carrying out the methods of the invention.

### ADVANTAGES OF THE INVENTION

With the monoclonal antibodies of the invention it is possible to monitor the detailed topology and proportion of target bacterial cells relative to other bacterial species during the course of plaque formation and the initiation and progression of carious lesions in a subject (e.g. with fluorescence microscopy). This in turn can lead to the development of improved treatment of dental caries. For example, antibodies of the invention can be conjugated with a regular or fluorescent dye. A solution containing such antibodies can be used to rinse a patient's mouth. The dyelinked antibodies can bind to the location of the dental caries. The dental caries image can be shown on a TV screen through a video or digital micro-camera.

With the fluorescent dye-linked monoclonal antibody and video imaging techniques, it is possible to label the bacteria at infection sites and thereby assist in detecting carious lesions at an early stage and in determining whether or not the lesion is active. This aids diagnosis, treatment and improves the management of dental health.

The monoclonal antibody based detection methods of the invention allows a rapid, accurate, and economic way to quantitatively measure the target bacteria in a subject, with significant advantages compared to current methods. As the first step towards development of effective and accurate caries risk assessment systems, we have described methods in this study that combine monoclonal antibodies with fluorometry techniques for detection and enumeration of target bacteria. These methods, especially flow cytometry, are able to rapidly detect the bacterium with high specificity and enumerate it with high accuracy. With these methods, it will be possible to process a large number of saliva samples in a short period of time at low cost. This will allow low cost, accurate assays to reevaluate the correlation between the salivary count of target bacteria and the presence and rate of progression of dental caries. Such assays can consists of monoclonal antibodies linked to a colloidal gold calorimetric system on test strips. The invention includes the use of a test system for rapid and simple assay of target bacteria by color change with simple immersion in fresh saliva. Such a method is suitable for use at a dentist's chairside as well as in the patient's household to assess dental caries risk. The accurate and objective assessment of dental caries risk state and/or caries activity state with any of these or with similar technologies will permit targeted preventive and curative treatment, thereby significantly improving human dental health.

The following examples are offered by way of illustration and not by way of limitation.

### Experimental

I. Materials and Methods

A. Bacterial Strains, Media, and Culture Conditions

Actinomyces bovis (ATCC 13683), A. denticolens (ATCC 43322), A. gerencseriae (ATCC 23860), A. israelii (ATCC 12102), A. meyeri (ATCC 35568), A. naeslundii (ATCC 12104, ATCC 49340, ATCC 19246, ATCC 27044, ATCC 43146), A. viscosis (OMZ 716, OMZ 722, OMZ 723, OMZ 724, OMZ 740, a kind gift from Dr. Rudolf Gmur), A. odontolyticus (ATCC 17929), A. viscosus (ATCC 15987), Fusobacterium nucleatum (ATCC 10953), Streptococcus mutans (ATCC 25175, UA 159), S. gordonii (ATCC 10558), S. sanguis (ATCC 10556) and S. sobrinus (ATCC 6715, ATCC 33478) were grown in Brain-Heart Infusion (BHI, DIFCO 0037-17) medium anaerobically (80% N₂, 10% CO₂, and 10% H₂ at 37° C.). Lactobacillus acidophilus (ATCC 4356), L. casei (ATCC 11578, ATCC 4646), L. rhamnosus (ATCC 9595), L. plantarum (ATCC 14917), L. salivarius (ATCC 11742), and L. oris (ATCC 49062) were grown anaerobically at 37° C. in LB Broth, Miller (LMB, DIFCO 0446-17) medium.

For in vitro dental plaque development, the standard simulated oral fluid (designated basal medium mucin, BMM, pH 7.0) was used. BMM contains 2.5 g/l partially purified pig gastric mucin (type III; Sigma Chemical Co., St Louis, Mo.), 10.0 g/l peptone (Oxoid, Unipath, Basingstoke, UK), 5.0 g/l trypticase peptone (BBL, Becton Dickinson, Md.), 5.0 g/l yeast extract (Difco Laboratories, Detroit, Mich.), 2.5 g/l KCl, 5 mg/l haemin, 1 mg/l menadione, 1 mM urea, 1 mM arginine, and 0.02% glucose.

B. Production and Screening of MAbs against A. naeslundii and L. casei

A. naeslundii (ATCC 12104) and L. casei (ATCC 11578) were grown to log phase in BHI and LMB medium respectively. The hybridomas for production antibodies against these two bacteria were raised using same procedure as reported previously. Shi et al. (1998) Hybridoma 17:363-371. The initial screening was performed with enzyme-linked immunoadsorbent assay (ELISA) assay as described previously, (Shi et al. (1998) supra) for detection of culture supernatants containing antibodies reactive with the corresponding bacteria. Supernatants with positive reactivity were then subjected to the immunoprecipitation assay (mixing 100 μl bacteria with 100 μl supernatant) to screen for those with strong positive reactivity. These supernatants were then used to test cross-reactivity with other bacteria (listed in Table 1).

C. Detection of A. naeslundii and L. casei with Fluorescence Microscopy

A. naeslundii or L. casei were suspended in either culture medium or 100 mM Phosphate buffer solution (PBS) (pH 7.4). 10 μl of the bacterial solution were mixed with 10 μl MAb-containing hybrodoma culture supernatant and incubated at RT for 5 min., 1 μl FITC linked goat anti-mouse IgG antibody was then added to the mixture. After an additional incubation for 10 min., the mixture was observed using phase-contrast microscopy and fluorescent microscopy.

D. Detection of A. naeslundii and L. casei with Flow Cytometry

The bacteria were labeled with FITC molecules as described above and analyzed with a Fluorescence-Activated Cell Sorter (FACS; Coulter EPICS elite flow cytometer, Miami, Fla.). Flow cytometry allows quantitative detection of bacteria that are labeled with FITC-linked MAbs according to their fluorescence intensity.

E. Labeling A. naeslundii and L. casei with CellTracker™ Orange CMTMR

A. naeslundii or L. casei were grown overnight at 37° C. anaerobically, together with CellTracker™ Orange CMTMR (Molecular Probes, Inc.) at a final concentration of 1 μM to specifically label these strains fluorescent orange. The labeled bacteria were then washed 3 times with PBS before testing.

Collection of Unstimulated and Stimulated Human Saliva

Unstimulated saliva samples were collected by asking participating human subjects to spit saliva into disposable plastic cups. For collection of stimulated saliva samples, participating human subjects chewed a piece of paraffin wax for 30 seconds before spitting saliva into disposable plastic cups. If the saliva samples could not be processed right after collection, 1% formaldehyde was often used to fix saliva samples.⁽⁹⁾ This procedure enables accurate enumeration of salivary bacteria for several weeks after collection. For these experiments, 0.45 ml of the collected saliva samples were transferred to 1.5 ml Eppendorf test tubes containing 0.05 ml 10% formaldehyde using a plastic pipette and mixed for three seconds. Bacteria-free salivary solutions were produced as follows: the collected stimulated and unstimulated saliva from different human subjects were centrifuged at 5000×g for 15 min to remove the majority of bacteria and particles. The supernatant was then sterilized via filtering through a 0.2 μm filter.

F. Development of Dental Plaque In Vitro

Artificial dental plaque was developed according to an in vitro human bacterial plaque growth model system. Wolinsky et al. (2000) J. Clin. Dent. 11:53-59. A cover glass was incubated with pooled stimulated human saliva anaerobically at 37° C. for 5 h. After being washed three times with a washing buffer (0.01 M K₃PO₄, 1.0 mM CaCl₂, 0.1 mM MgCl₂, pH 7.0), the cover glass was then cultivated in BMM to allow growth of bacteria that attached to the conditioned cover glass. Sissions et al. (1991) J. Dent. Res. 70:1409-1416. The above process was repeated until a mature dental plaque was formed.

G. Detection of A. naeslundii and L. casei within Artificial Dental Plaque

All bacteria within mature artificial dental plaque were stained by incubating the plaque with 1 μM SYTO13 green fluorescent nucleic acid stain (Molecular Probes, Inc.) at room temperature (RT) for 30 min. After washing with the washing buffer three times, the dental plaque was incubated at RT for 30 min with 50 μl culture supernatant of a hybridoma cell line producing the respective antibody. The plaque was then again washed three times with washing buffer before incubation with 10 μl of Alexa Fluor® 568 conjugated goat anti-mouse IgG (1 μg/ml) at RT for 30 min. After three more times washing to remove excess antibody molecules, the labeled dental plaque was examined with Confocal Laser Scanning Microscopy (CLSM).

II. Results

A. Production and Isolation of MAbs Against A. naeslundii and L. casei

Three BALB/c mice were immunized with formalinized A. naeslundii (ATCC 12104) and L. casei (ATCC 11578) respectively, and used for production of MAbs. 978 mature hybridomas for A. naeslundii and 742 for L. casei were obtained. All mature hybridoma supernatants were screened with ELISA, and 235 supernatants were found to have positive reactivity with A. naeslundii and 121 supernatants were shown to have positive reactivity with L. casei. Further immunoprecipitation assays identified 13 supernatants that exhibited strong positive reactivity against A. naeslundii and 7 supernatants showed strong positive reactivity against L. casei. These culture supernatants were used to test cross-reactivity with a variety of other oral bacteria listed in Table 1. One supernatant each was identified that had the highest positive reactivity with A. naeslundii and L. casei respectively, yet did not have any significant cross-reactivity with the other bacteria tested. The corresponding antibodies against these two species were named SWLA4 for A. naeslundii and SWLA5 for L. casei. Subclass isotype analysis indicated that both MAbs are IgG.

B. Detection of A. naeslundii and L. casei with FACS

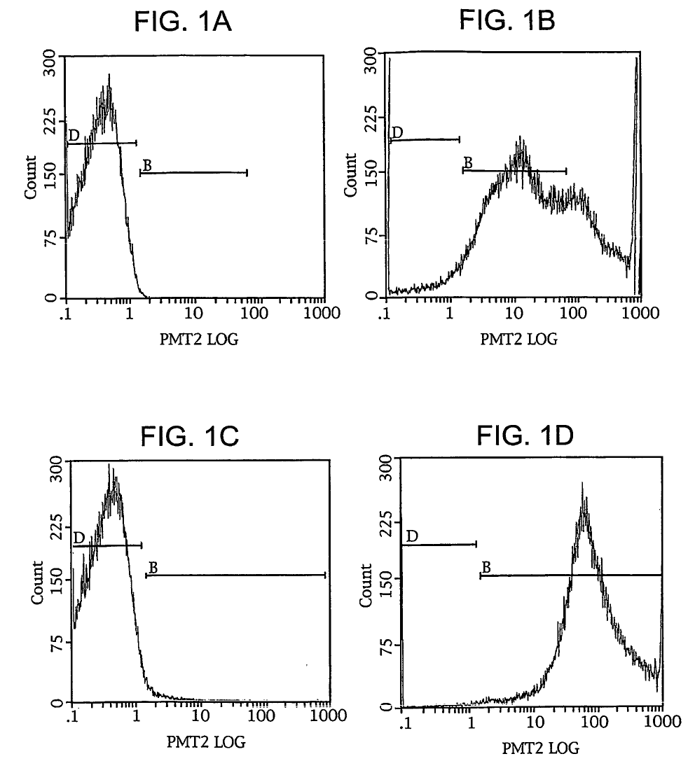

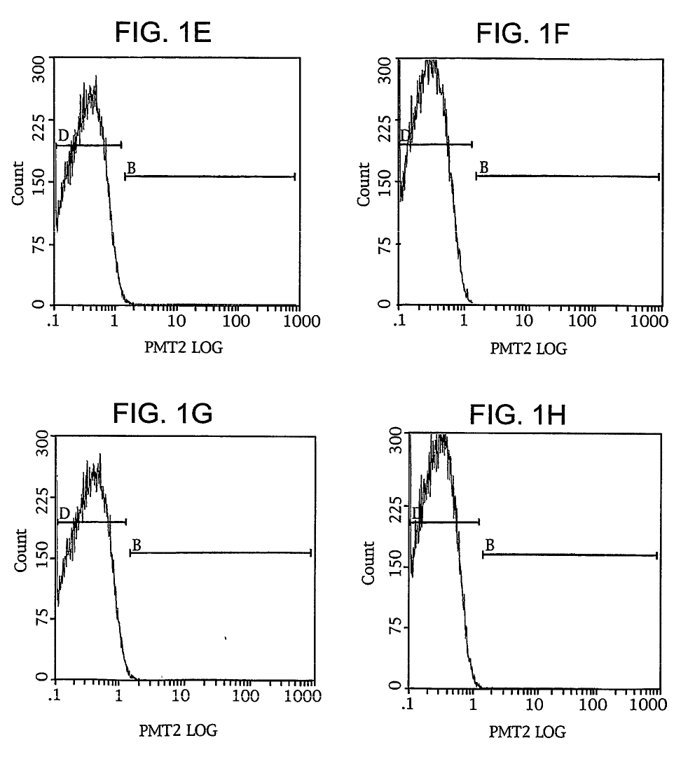

A fluorescence-activated cell sorter (FACS) is able to detect particles in a solution and to separate them based on their fluorescence intensities. In this study, FACS was used to further analyze the specificity of the MAbs against A. naeslundii and L. casei. Both species were individually labeled with FITC as described in MATERIALS AND METHODS and efficiently detected with FACS (FIGS. 1a-d). A mixture consisting of nine other oral bacteria that were processed in identical ways using the same antibodies did not elicit any detectable signal (FIGS. 1e-h). However, FACS does not appear to be suitable for quantitative analysis of A. naeslundii and L. casei because these bacteria tend to form larger aggregates that would affect the accuracy of enumeration.

FIGS. 1a-h depict flow cytometry analysis of oral bacteria. X-axis is amount of FITC associated with bacterial cells, while Y-axis is number of bacteria. The bacterial mixture consists of A. israelii (ATCC 12102), A. meyeri (ATCC 35568), A. viscosus (ATCC 15987), Streptococcus mutans (ATCC 25175), S. sobrinus (ATCC 33478), L. acidophilus (ATCC 4356), L. salivarius (ATCC 11742), L. plantarum (ATCC 14917) and L. oris (ATCC 49062). (a) A. naeslundii (ATCC 12104) treated with FITC linked goat-anti-mouse IgG antibody only; (b) A. naeslundii treated with SWLA4 and FITC linked goat-anti-mouse IgG antibody; (c) L. casei (ATCC 11578) treated with FITC linked goat-anti-mouse IgG antibody only; (d) L. casei treated with SWLA5 and FITC linked goat-anti-mouse IgG antibody; (e) The bacterial mixture treated with FITC linked goat-anti-mouse IgG antibody only; (f) The bacterial mixture treated with SWLA4 and FITC linked goat-anti-mouse IgG antibody; (g) The bacterial mixture treated with FITC linked goat-anti-mouse IgG antibody only; (h) The bacterial mixture treated with SWLA5 and FITC linked goat-anti-mouse IgG antibody. Similar results were obtained with strains OMZ 716, OMZ 722, OMZ 723, OMZ 724, OMZ 740 for A. naeslundii, and ATCC 4646 for L. casei

C. Detection of A. naeslundii and L. casei with Fluorescence Microscopy

Fluorescent microscopy was used to test the sensitivities and specificities of SWLA4 and SWLA5. Pure A. naeslundii or L. casei cultures were FITC-labeled with either SWLA4 or SWLA5, respectively, according to the procedures described in MATERIALS AND METHODS. The phase contrast and fluorescent image were acquired. Superimposition of both images demonstrated that virtually all bacteria were fluorescent green labeled, indicating high sensitivity of the antibodies in detecting their corresponding bacteria.

Next, we performed the following experiments to test the specificity of the antibodies: A. naeslundii and L. casei were grown in the presence of an orange fluorescent dye as described in MATERIALS AND METHODS. The labeled bacteria (fluorescent orange) were then individually mixed with other unlabeled oral bacterial species including A. israeli, A. meyeri, A. viscosus, S. mutans, S. sobrinus, L. acidophilus, L. salivarius, L. plantarum, and L. oris. The mixtures (containing either fluorescent orange A. naeslundii or fluorescent orange L. casei) were then labeled with FITC (fluorescent green) conjugated SWLA4 or SWLA5, respectively. For both A. naeslundii and L. casei, only fluorescent orange bacteria were also labeled with FITC (fluorescent green) and vice versa, demonstrating that the specificity of the MAbs even in the presence of a variety of other bacteria is about 100%. The data suggest that these antibodies would allow accurate enumeration of both cariogenic bacteria in high sensitivity and specificity. Similar results were obtained with strains OMZ 716, OMZ 722, OMZ 723, OMZ 724, OMZ 740.

D. SWLA Antibodies Effectively Detect A. naeslundii and L. casei in Saliva

In our previous study, it has been shown that S. mutans in saliva can be successfully enumerated with MAbs based detection techniques. Gu et al. (2002) Hybridoma and Hybridomics 21: 225-232. We used the same strategy in this study to evaluate the abilities of SWLA4 and SWLA5 monoclonal antibodies to detect A. naeslundii and L. casei in saliva. To test whether salivary solutions would interfere with the detection of bacteria in saliva, stimulated and unstimulated saliva were collected, and filter-sterilized. Known numbers of bacteria were resuspended in these bacteria-free salivary solutions that were prepared as described above, as well as in phosphate buffer saline (PBS). SWLA antibodies and fluorescent microscopy were used to detect and quantify bacteria in these salivary solutions. As shown in Table 2, SWLA antibodies can effectively detect A. naeslundii and L. casei in these salivary solutions with the same sensitivity as in PBS, indicating that components present in salivary solutions did not affect the specific binding between SWLA4 and SWLA5 antibodies and their cognate bacterial cells.

The centrifugation and filtration procedure used to prepare salivary solutions removes big particle in addition to bacteria from the whole saliva sample. These “particles” could potentially interfere with the specific binding of the antibody to bacterial cells. To address this concern we added a known amount of A. naeslundii or L. casei respectively to various saliva samples and compared the difference before and after addition of bacteria. As shown in Table 3, bacteria cells added to whole saliva were accurately detected by SWLA antibodies with fluorescent microscopy as previously demonstrated already for S. mutans-specific SWLA1-3. Gu et al. (2002), supra. Apparently, components in whole saliva also did not affect the specific binding between SWLA4 and SWLA5 antibodies and A. naeslundii or L. casei.

Since monoclonal antibodies may recognize not only living bacteria, but also dead bacteria with intact cell envelopes, we explored the possibility whether SWLA antibodies may still be able to recognize bacteria cells in saliva fixed with formaldehyde. Consistent with results obtained for the SWLA1-3 antibodies against S. mutans,⁽⁹⁾ SWLA4 and SWLA5 are also able to recognize 1% formaldehyde fixed A. naeslundii and L. casei cells in saliva at the same sensitivity as living bacteria cells (Table 3). This will allow dentists to fix saliva samples at chairside and ship to laboratory for processing at a later time.

E. A Profile of Salivary A. naeslundii and L. casei in a Children Population

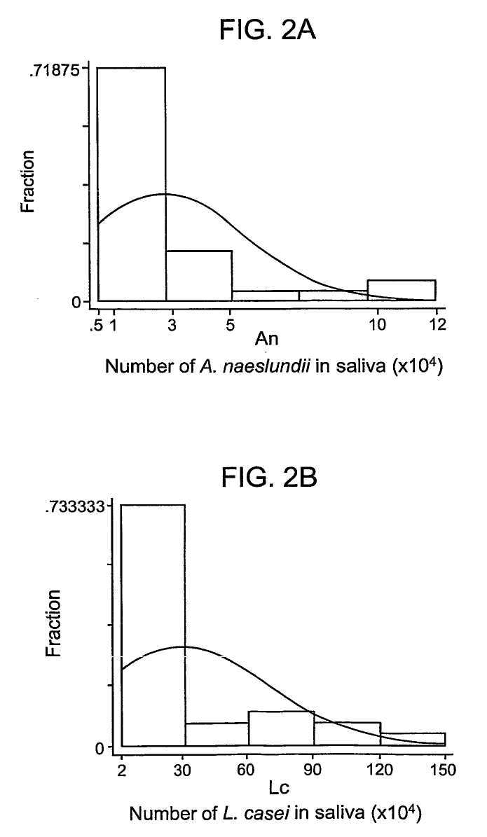

We analyzed 100 saliva samples collected from children between age 2 and 16 using SWLA antibodies based methods. FIGS. 2a and 2b show the profile of salivary A. naeslundii and L. casei among these children. The number of A. naeslundii in tested saliva samples varies from 0.5×10⁴ to 4.8×10⁵ cells/ml (FIG. 2a) and the number of L. casei ranges from 1×10⁴ to 1.2×10⁶ cells/ml (FIG. 2b). The variation in the number of L. casei is similar to what we observed for S. mutans in human saliva which ranges from less than 1×10⁴ to 3.6×10⁶ cells/ml, while as a comparison, the number of salivary A. naeslundii seems to have less variation.

FIGS. 2a and 2b depict distribution of salivary A. naeslundii (genospecies 1) and L. casei counts within a human population. The data were based on saliva samples collected from 100 children aged from 2-16. The unstimulated saliva samples were collected and fixed at the dentists' chairside and shipped to UCLA for processing (as described in MATERIALS AND METHODS). (a) Distribution of salivary A. naeslundii (genospecies 1) counts; (b) Distribution of salivary L. casei counts.

Since the levels of S. mutans, L. casei and A. naelundii in saliva are all considered to be associated with dental caries, we wanted to further explore potential correlations between salivary counts of these three bacteria. Pearson correlation analysis was used to examine the salivary counts of A. naeslundii, L. casei and S. mutans in these 100 saliva samples. The results indicate a statistically significant positive correlation between the salivary counts of L. casei and S. mutans (ρ=0.5), but not A. naeslundii and S. mutans (ρ=0.05). For A. naeslundii and L. casei, there is a relatively weak positive correlation, since the Pearson correlation coefficient is around 0.2 (ρ=0.2).

F. Detection of A. naeslundii and L. casei within Artificial Dental Plaque

The MAb-based detection techniques could detect the corresponding bacteria not only in saliva but also within dental plaque. The nucleic acid stain SYTO13 (fluorescent green) was used to stain entire bacteria population within dental plaque as described in MATERIAL AND METHODS. The plaques were then stained with SWLA antibodies in conjunction with Alexa Fluor® 568 (fluorescent red) conjugated secondary antibodies. Images of a side-view (XZ-plane) of the distribution of A. naeslundii within an artificial dental plaque; and a XY-plane of the same field were made. The images show that A. naeslundii cells are scattered among all layers within the artificial dental plaque. The XZ-plane view and XY-plane view of the distribution of L. casei within an artificial dental plaque. Only few L. casei cells were observed, most of them being dispersed among the top layers within the artificial dental plaque, suggesting that L casei was not a major bacterium in this type of dental plaque.

III. Discussion

Dental caries is considered as a bacteria-dependent multifactor disease. The profile of caries distribution within population is very uneven thus making it very meaningful to identify those at high risk. Epidemiological studies indicate a possible association between the level and proportion of cariogenic bacteria in saliva or plaque and the incidence of dental caries. This association suggests that with proper bacterial detection methods, people at high risk for dental caries may be diagnosed. To validate the utility of microbial identification and enumeration in saliva for caries diagnosis and risk assessment, a properbacterial detection method is necessary. Since the beginning of monoclonal antibody era in 1975, monoclonal antibody techniques have been widely applied to diagnostic and therapeutic fields. Using hybridoma techniques, species-specific monoclonal antibodies can be raised against unique components on bacterial surface. The MAbs can be linked to various detection systems such as fluorescent dyes, colorimetric or coagglutination reagents, allowing rapid presentation of specific detection results. In our previous study, monoclonal antibodies against S. mutans were developed and monoclonal antibodies (MAbs) based techniques were shown to have high specificity and sensitivity. This previous study and the data presented here also demonstrate that the MAb-based techniques represent simple and reliable enumerating methods for the cariogenic bacteria and will be useful tools for clinical diagnosis and risk assessment.

Since multiple bacterial species are involved in dental caries, understanding the whole profile of cariogenic bacterial species would be particularly useful for validating the utility of microbial identification and enumeration in saliva for caries diagnosis and risk assessment. Through this study, we are now able to assess the most frequently detected bacteria in caries lesions as representative strains of three major cariogenic groups of bacteria: S. mutans for the mutans streptococci, A. naeslundii for the actinomyces group and L. casei for the lactobacilli.

In this study, we used whole cells of A. naeslundii and L. casei as antigens to ensure that raised antibodies can recognize surface structures of the individual bacterial species. We have also produced and screened a large amount of hybridomas that allow us to obtain highly species-specific diagnostic antibodies. One monoclonal antibody was identified for each species. These monoclonal antibodies (SWLA4 for A. naeslundii and SWLA5 for L. casei) were shown to have high sensitivities and specificities in quantitative identification of A. naeslundii and L. casei in human saliva. These antibodies should have a great potential to become a versatile tool for general assessment of bacterial profiles in saliva samples.

Our study showed a great variation in salivary L. casei counts within a human population. Among 100 human saliva samples tested, we found that L. casei varied from 1×10⁴ to 1.2×10⁶ cells/ml, similar to the range of salivary S. mutans (1×10⁴ to 3.6×10⁶ cells/ml).⁽⁹⁾ The salivary level of A. naeslundii is generally lower than that of L. casei or S. mutans. Among 100 human saliva samples tested, the number of A. naeslundii ranged from less than 0.5×10⁴ to 4.8×10⁵ cells/ml. Since the levels of S. mutans, L. casei and A. naelundii in saliva are considered to be associated with dental caries, the correlations between these three bacterial species in saliva were obtained. The correlations between A. naeslundii, L. casei and S. mutans were evaluated with Pearson correlation analysis. The results indicate a statistically significant positive correlation between the salivary numbers of L. casei and S. mutans (Pearson correlation coefficient ρ=0.5), but not A. naeslundii and S. mutans (ρ=0.05). For A. naeslundii and L. casei, there is a fairly weak positive correlation, since the Pearson correlation coefficient is around 0.2 (ρ=0.2). The positive correlation between L. casei and S. mutans in saliva may be due to the fact that both bacteria are very acidogenic and aciduric. On the contrary, A. naeslundii is a very early colonizer, not as aciduric as the other two species and is typically superseded by more acidogenic and acidoduric species such as S. mutans and L. casei. This might explain the lack of a positive correlation between A. naeslundii and S. mutans, and the very weak correlation between A. naeslundii and L. casei.

A previous study found that the proportion of A. naeslundii was significantly higher in initial lesions than in advanced lesions. The sound exposed root surfaces from which A. naeslundii was isolated yielded significantly lower numbers of lactobacilli than the surfaces from which A. naeslundii were not isolated. In addition, subjects without root-surface caries or restorations, as compared with subjects with root-surface caries with or without restorations, were characterized by having a lower prevalence and proportion of mutans streptococci and a higher prevalence and proportion of A. naeslundii in plaque on sound root surfaces. Furthermore, it's been known that lactobacilli are associated more with carious dentine and the advanced carious lesions, whereas S. mutans has been considered as a major cariogenic bacterium involved in the initiation and progression of dental caries. These relationships indicate that the quantification of S. mutans, A. naeslundii and L. casei on tooth surfaces can be used as a diagnostic tool for caries. In this study, we showed that the MAbs that are highly specific for the three species of interest can localize the corresponding bacteria within dental plaque in situ, thus providing another potential way of caries risk assessment and early diagnosis by examining the distributions of these three cariogenic bacteria on tooth surface.

A. viscosus and A. naeslundii were previously classified as two separate species. However, they were recently re-classified as two genospecies of A. naeslundii according to their antigenic relationships among oral actinomyces isolates using agglutination and immunoblofting properties as a marker. Putnins and Bowden (1993) J. Dent Res. 72:1374-1385. The strains used in this study as A. naeslundii, ATCC 12104, OMZ 716, OMZ 722, OMZ 723, OMZ 724, OMZ 740, belong to genospecies 1; and strains used in this study as A. viscosus, ATCC 49340, ATCC 19246, ATCC 27044, ATCC 43146, belong to genospecies 2. SWLA4 produced for A. naeslundii in this study specifically detects genospecies 1 and has no cross reactivity with genospecies 2 (Table 1). Thus it can be used to classify the genospecies of A. naeslundii and study the special features of genospecies 1.

In summary, we have demonstrated that SWLA4 and SWLA5 antibodies can be used to effectively and accurately detect A. naeslundii (genospecies 1) and L. casei in saliva and dental plaque. Together with the SWLA antibodies against S. mutans we produced previously, we are now able to detect representative strains from three major cariogenic groups, mutans streptococci, actinomyces and lactobacilli. This provides a new opportunity and new tool for dental researchers to confirm whether A. naeslundii; L. casei and S. mutans levels in saliva or on tooth surface correlate with caries risk state and/or caries activity status.

TABLE 1
Oral bacterial strains and their reactivities with monoclonal antibodies.

Cross-reactivity

| Bacterial species | Strain names | SWAL4 | SWAL5 |
|---|---|---|---|
| L. casei | ATCC 11578 | − | + |
| | ATCC 4646 | − | + |
| L. rhamnosus | ATCC 9595 | − | − |
| L. acidophilus | ATCC 4356 | − | − |
| L. salivarius | ATCC 11742 | − | − |
| L. plantarum | ATCC 14917 | − | − |
| L. oris | ATCC 49062 | − | − |
| A. naeslundii | ATCC 12104 | + | − |
| | OMZ 716 | + | − |
| | OMZ 722 | + | − |
| | OMZ 723 | + | − |
| | OMZ 724 | + | − |
| | OMZ 740 | + | − |
| A. viscosus | ATCC 19246 | − | − |
| | ATCC 27044 | − | − |
| | ATCC 43146 | − | − |
| | ATCC 15987 | − | − |
| A. israelii | ATCC 12102 | − | − |
| A. gerenseriae | ATCC 23860 | − | − |
| A. meyeri | ATCC 35568 | − | − |
| A. odontolyticus | ATCC 17929 | − | − |
| A. denticolens | ATCC 43322 | − | − |
| A. bovis | ATCC 13683 | − | − |
| S. mutans | ATCC 25175 | − | − |
| | UA 159 | − | − |
| S. rattus | ATCC 19645 | − | − |
| S. sobrinus | ATCC 33478 | − | − |
| | ATCC 6715 | − | − |
| S. sanguis | ATCC 10556 | − | − |
| S. gordonii | ATCC 10558 | − | − |
| P. gingivalis | ATCC 33277 | − | − |
| F. nucleatum | ATCC 10953 | − | − |

Immunoprecipitation, and fluorescent microscopy was used to screen the cross-reactivity of antibodies SWLA4 and SWLA5 with various bacterial strains. See MATERIALS AND METHODS for experimental procedures.

TABLE 2
Detection of A. naeslundii and L. casei cells within various solutions using
SWLA antibodies-based techniques.

| | | Number of bacteria detected | | |
|---|---|---|---|---|
| | Number of | | Filtered unstimulated | Filtered stimulated |
| Bacterial strains | bacteria added | PBS | saliva | saliva |
| A. naeslundii | 1.10 × 10⁶ | (1.12 ± 0.04) × 10⁶ | (1.14 ± 0.05) × 10⁶ | (1.11 ± 0.05) × 10⁶ |
| (ATCC 12104) | 1.20 × 10⁵ | (1.23 ± 0.05) × 10⁵ | (1.26 ± 0.07) × 10⁵ | (1.20 ± 0.06) × 10⁵ |
| | 1.80 × 10⁴ | (1.86 ± 0.07) × 10⁴ | (1.88 ± 0.09) × 10⁴ | (1.81 ± 0.11) × 10⁴ |
| L. casei | 2.50 × 10⁶ | (2.48 ± 0.03) × 10⁶ | (2.51 ± 0.04) × 10⁶ | (2.52 ± 0.03) × 10⁶ |
| (ATCC 11578) | 2.40 × 10⁵ | (2.39 ± 0.05) × 10⁵ | (2.40 ± 0.07) × 10⁵ | (2.39 ± 0.07) × 10⁵ |
| | 2.90 × 10⁴ | (2.92 ± 0.06) × 10⁴ | (2.91 ± 0.08) × 10⁴ | (2.92 ± 0.09) × 10⁴ |

A known number of A. naeslundii or L. casei cells (enumerated with a bacterial counting chamber) were resuspended in phosphate buffer solution (PBS) or various salivary solutions, treated with FITC-conjugated SWLA antibodies and examined with fluorescent microscopy. Data shown represent the means and standard deviations calculated based on the bacterial counting results in salivary solutions from three different subjects (salivary solutions were collected and prepared as described in MATERIALS AND METHODS). The bacterial counts in PBS are the average of triplicate counts of the same sample. Strains ATCC 12104 and ATCC 11578 were used for the data shown in the table. Similar results were obtained with strains OMZ 716, OMZ 722, OMZ 723, OMZ 724, and OMZ 740 for A. naeslundii, and ATCC 4646 for L. casei.

TABLE 3
SWLA antibodies specifically and accurately detect A. naeslundii
and L. casei in saliva with or without 1% formaldehyde

| | | | | Number of |
|---|---|---|---|---|
| | | Number | Number | bacteria in |
| | | of bacteria in | of bacteria in | saliva (After*) |
| Bacterial strain | | saliva | saliva | fixed with 1% |
| added | Subject | (Before) | (After*) | formaldehyde |
| A. naeslundii | 1 | 3.00 × 10⁴ | 0.98 × 10⁶ | 1.19 × 10⁶ |
| (ATCC 12104) | 2 | 7.00 × 10⁴ | 1.10 × 10⁶ | 1.23 × 10⁶ |
| | 3 | 4.00 × 10⁴ | 1.13 × 10⁶ | 0.97 × 10⁶ |
| L. casei | 1 | 1.20 × 10⁶ | 2.20 × 10⁶ | 2.11 × 10⁶ |
| (ATCC 11578) | 2 | 1.40 × 10⁶ | 2.43 × 10⁶ | 2.22 × 10⁶ |
| | 3 | 6.10 × 10⁵ | 1.54 × 10⁶ | 1.78 × 10⁶ |

*After addition of 1 × 10⁶ A. naeslundii or L. casei cells saliva samples.

1×10⁶ A. naeslundii or L. casei cells (enumerated with a bacterial counting chamber) were resuspended in unfiltered saliva collected from three different subjects with or without 1% formaldehyde fixation, treated with FITC-conjugated SWLA4 and SWLA5 respectively and examined with fluorescent microscopy (as described in MATERIALS AND METHODS). Data shown are the average of triplicate counts of the same sample with errors less than 10%. Strains ATCC 12104 and ATCC 11578 were used for the data shown in the table. Similar results were obtained with strains OMZ 716, OMZ 722, OMZ 723, OMZ 724, and OMZ 740 for A. naeslundii, and ATCC 4646 for L. casei.
IV. Specific Representative Applications
A. SWAL4 and SWAL5 antibodies are conjugated with colloidal gold particles. The resultant conjugated antibody reagents are employed in chair-side/bed-side instant detection kits for cariogenic bacteria.
B. SWAL4 and SWAL5 antibodies are conjugated with color latex beads. The resultant conjugated antibody reagents are employed in chair-side/bed-side instant detection kits for cariogenic bacteria.
C. SWAL4 and SWAL5 antibodies are conjugated with fluorescent dyes. The resultant conjugated antibodies are employed for detection of cariogenic bacteria in saliva or dental plaques.
D. SWAL4 and/or SWAL5, and/or S. mutans specific antibodies, as described earlier, are each conjugated to different and distinguishable fluorescent dyes, where the resultant fluorescent labeled antibodies find use in multiplex detection applications for the detection of two or more different cariogenic bacteria in a single sample at the same time.
E. The genes encoding for SWAL4 and/or SWAL5 antibodies are cloned and used for production of humanized antibodies against these cariogenic bacteria for use in passive vaccination against these cariogenic bacteria in humans. Similar approaches are applied to pets and other animals.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. An antibody that recognizes a cell surface antigen of a target cariogenic bacterium chosen from an actinomyces and a lactobacillus species, wherein said antibody has high specificity and sensitivity for its target bacterium.

2. The antibody according to claim 1, wherein said target bacterium is an actinomyces species bacterium.

3. The antibody according to claim 2, wherein said actinomyces species is Actinomyces naeslundii genospecies 1.

4. The antibody according to claim 3, wherein said antibody does not cross-react with Antinomyces naeslundii genospecies 2.

5. The antibody according to claim 1, wherein said target bacterium is a lactobacillus species bacterium.

6. The antibody according to claim 5, wherein said lactobacillus species is Lactobacillus casei.

7. The antibody according to claim 1, wherein said antibody is produced using a whole cell immunogen.

8. The antibody according to claim 1, wherein said antibody is a monoclonal antibody.

9. The antibody according to claim 8, wherein said antibody is chosen from SWLA4 and SWLA5.

10. An antibody that binds to Actinomyces naeslundii with at least substantially the same sensitivity and specificity as SWLA4.

11. An antibody that binds to Lactobacillus casei with at least substantially the same sensitivity and specificity as SWLA5.

12. A binding fragment or mimetic thereof of an antibody according to claim 1.

13. A method for detecting the presence of a cariogenic bacterium in a sample, said method comprising:
(a) contacting said sample with an antibody according to claim 1 or a binding fragment or mimetic thereof; and
(b) detecting the presence of any resultant binding complex between said cariogenic bacterium and said antibody, binding fragment or mimetic thereof to detect the presence of said cariogenic bacterium in said sample.

14. The method according to claim 13, wherein said sample is a physiological sample.

15. The method according to claim 14, wherein said physiological sample is saliva.

16. The method according to claim 14, wherein said physiological sample is dental plaque.

17. The method according to claim 13, wherein said antibody, binding fragment or mimetic thereof is stably associated with a solid support.

18. A device for use in determining the presence of a cariogenic bacterium in a sample, said device comprising:
an antibody according to claim 1 or binding fragment or mimetic thereof stably associated with the surface of a solid support.

19. A cell that secretes an antibody according to claim 1.

20. The cell according to claim 19, wherein said cell is a hybridoma cell.

21. A method of treating a host suffering from a condition resulting from the presence of cariogenic bacteria, said method comprising:
administering to said host an effective amount of an antibody according to claim 1 or a binding fragment or mimetic thereof.

22. The method according to claim 21, wherein said antibody, binding fragment or mimetic thereof is conjugated to a therapeutically active agent.

23. A pharmaceutical preparation comprising an antibody according to claim 1 or a binding fragment or mimetic thereof.

24. A kit for use in detecting the presence of a cariogenic bacterium in a sample, said kit comprising:
at least one antibody according to claim 1, or a binding fragment or mimetic thereof; and
instructions for using said antibody to detect the presence of said cariogenic bacterium.

25. The kit according to Clam 24, wherein said kit comprises at least a first antibody that binds to an actinomyces bacterium and a second antibody that binds to a lactobacillus bacterium.

26. An antibody according to claim 1 or a binding fragment or mimetic thereof conjugated to a detectable label.

27. The antibody according to claim 26, wherein said detectable label is a colloidal label.

28. The antibody according to claim 26, wherein said detectable label is a latex bead.

29. The antibody according to claim 26, wherein said detectable label is a fluorescent label.

30. A nucleic acid present in other than its natural environment that encodes an antibody according to claim 1.
